(19) United States
(12) Patent Application Publication
(10) Pub. No.: US 2012/0270933 A1
(43) Pub. Date: Oct. 25, 2012

(54) LIQUID STATIN FORMULATION

(75) Inventors: Ken Phelps, Leawood, KS (US); Jabar Qasem, Madison, AL (US); Lynn Gold, Cincinnati, OH (US)

(73) Assignee: MADEIRA THERAPEUTICS, Leawood, KS (US)

(21) Appl. No.: 13/538,904

(22) Filed: Jun. 29, 2012

Related U.S. Application Data
(63) Continuation of application No. 13/203,164, now abandoned.
(60) Provisional application No. 61/155,018, filed on Feb. 24, 2009.

(51) Int. Cl. A61K 31/366; A61K 47/14; A61K 47/22; A61K 47/18; A61K 47/24; A61P 3/00; A61K 47/12; A61K 47/46; A61K 47/20; A61K 47/26; A61K 47/02; A61K 47/10; A61K 47/40

(52) U.S. Cl. 514/460; 514/772; 514/786; 514/784; 514/777; 514/783; 514/782; 514/770; 514/769

(57) ABSTRACT

The present invention relates to compositions and methods for liquid statin products suitable for use in a person or animal. The invention provides stable liquid formulations containing a statin and at least one solubilizer. Methods for the oral administration of statin formulations are also provided by the invention.

### FIELD OF THE INVENTION

The present invention relates to methods and compositions for liquid statin products suitable for administration to humans or animals. Specifically, the invention provides liquid formulations containing a statin and methods for the oral administration of the formulations to children, adolescents, and animals.

### BACKGROUND OF THE INVENTION

Heterozygous familial hypercholesterolemia (HeFH) is a common monogenetic disorder characterized by defective low density lipoprotein cholesterol (LDL-C) receptors on the surface of hepatocytes, which leads to severely elevated levels of plasma LDL-C from birth onwards, and causes premature atherosclerosis and cardiovascular disease (CVD). Heterozygous familial hypercholesterolemia patients often exhibit serum cholesterol levels around 400 mg/dL (normal levels are below 200 mg/dL). The identification and management of HeFH in children and adolescents is highly desirable. If untreated, about 50% of males and females will develop CVD before the age of 60.

Functional and morphological changes of the blood vessel wall have been documented in children with HeFH. These changes indicate that the atherosclerotic process has already been initiated early in childhood. Indeed, children with HeFH are characterized by impaired endothelial function and increased intima-media thickness (IMT). As a sequel to these observations, myocardial ischemia and coronary artery stenoses have been documented in young adults with this disorder. Because functional and morphological arterial wall changes are already present in these children, statin treatment should be considered for every child diagnosed with HeFH. The early onset of atherosclerosis in patients with HeFH stresses the need to initiate statin therapy at a young age in children with this disorder.

In 2008 the American Academy of Pediatrics (AAP) recommended initial treatment with statins in children 8 years of age and older with a low density lipoprotein (LDL) concentration of either 190 mg/dL or more; 160 mg/dL with a family history of early heart disease or two additional risk factors present; or 130 mg/dL and diabetes mellitus. These recommendations are, in part, for the overall safe and efficacious use of statins in this population.

Several recent randomized, controlled clinical trials established both efficacy and safety of statin therapy in children aged 8 to 18 years old with HeFH for periods ranging from 12 to 104 weeks. In the studies reductions of LDL-C were quite similar to the reductions achieved in adults. The reported clinical trials spanned the age range of pubertal development, and had no impact on sexual or physical maturation.

Most statin medications are available either as tablets, capsules, or solutions for injection. An individual may have difficulty swallowing the usual solid dosage form, and daily injections are difficult to administer. Further, the tablets or capsules must be cut into pieces to yield the lower dosages that children generally require. It is known that “pill splitting” can adversely affect dosage accuracy and the stability of medications. Further, when pill splitting is used, either a crushed tablet or contents of the capsule generally must be mixed with solid food or drink to make them palatable for a child to ingest. Hence, it is desirable to have a liquid statin formulation, but such formulations are not available due to poor solubility or insolubility.

Difficulty in adjusting the statin dose is one of the problems encountered when treating children. Current approved labeling for statin use in children indicates that doses should be individualized according to the recommended goal of therapy. For children it is recommended that stepped titration up to the maximum recommended dose be performed until target LDL levels are achieved, or there is evidence of toxicity. Having a statin oral formulation provides flexibility to customize the dose, providing the ability to individualize therapy according to the specific recommended goal.

### SUMMARY OF THE INVENTION

The invention provides liquid formulations that include a solubilized statin and are suitable for oral administration to people, as well as, animals. These formulations are useful for lowering total cholesterol and the treatment of diseases that are associated with high cholesterol, such as HeFH, or cardiovascular disease. These liquid formulations are useful for treating children, adolescents, and other individuals to whom tablet or capsule formulations are difficult or impractical to administer or whose dosage is not available in solid form and should be individualized. They may also be used in treating animals, particularly companion animals. A preferred liquid formulation includes simvastatin. A preferred combination formulation includes simvastatin and atorvastatin.

The invention provides liquid formulations comprising 0.05-10% weight to weight (w/w) of a statin, such as simvastatin or a combination of statins such as simvastatin and atorvastatin. Preferred formulations of the invention have 0.05-2.5% w/w of statin, and more preferred liquid formulations have about 0.2% w/w statin. Alternatively, the amount of statin in a formulation may be expressed in mg/ml. Liquid formulations of the invention will comprise 0.01-25 mg/ml of statin, preferably formulations will include 1-5 mg/ml of statin, more preferably formulations include about 2 mg/ml of statin. Higher concentrations may be desirable so that volume/dose may be reduced. The total amount of statin in a formulation may be due to a single statin or a combination of statins.

Combinations of statins may, for example, include one or more Type I statins, Type II statins, or combinations thereof. Herein, Type I statins have a substituted decalin-ring and include lovastatin, mevastatin, pravastatin, and simvastatin. Preferred Type I statins include pravastatin, and simvastatin. Type II statins typically have a fluorophenyl group in place of the butyrl group that is present in Type I statins. Exemplary Type II statins include atorvastatin, cerivastatin, fluvastatin, rosuvastatin, and pitavastatin. Preferred Type II statins include atorvastatin, and pitavastatin. Statins may also be present in the formulations in combinations of more than one form, i.e. a statin may be present as an acid (e.g. carboxylic acid), salt (including calcium, sodium, potassium, and magnesium salts), or neutral (closed lactone ring) form. For example, one exemplary formulation includes simvastatin in a neutral or closed lactone ring form and in a sodium salt form. Similarly, a formulation may include a combination of pitavastatin, which is a double molecule calcium salt, and pravastatin, which has a closed lactone ring. Alternatively, sodium, potassium, or calcium salts of simvastatin and pravastatin may be combined together in a formulation. Those of skill in the art will recognize that the desired combination of statins will depend upon the proposed end user, the objective of the treatment, and the solubility of the combination.

Formulations of the invention also include a vehicle (i.e. a solubilizer or solubilizing agent) to solubilize the statin. While many solubilizers are known to those of skill in the art, data show that certain vehicles, or combinations thereof, are more suitable than others. It is envisioned that liquid formulations of the invention may include one or more of the following vehicles: propylene glycol, minerals, propylene glycolmonostearate, propylene glycol alginate, natural glycerine, niacin, synthetic glycerine, vitamins, sorbitol, alcohols, myristyl alcohol, carboxymethylcellulose, labrasol, copovidone, Captex 355, croscarmellose sodium, polyethylene glycol (PEG) 400, PEG 1000, PEG 1450, PEG 1540, crospovidone, ethyl cellulose, aqueous polysorbate 20, aqueous polysorbate 40, aqueous polysorbate 60, aqueous polysorbate 80, cellulose, oxidized cellulose, polyoxyl 10 oleoyl ether, cellulose sodium phosphate, polyoxyl 20 cetostearyl, hyopromellose, poloyxyl 35 castor oil, polyoxyl 40 hydrogentated castor oil, polyoxyl 40 stearate, poloxyl lauryl ether, poloxyl oleate, poloxyl stearyl ether, or any combination thereof. Preferred vehicles include propylene glycol, polyethylene glycol, PEG 400, glycerine, and combinations of glycerine with either propylene glycol or polyethylene glycols of various molecular weights.

Preferred embodiments of oral solutions of the invention also include an antioxidant, flavoring, preservative, or a combination thereof. More preferably, oral solutions include all three elements (i.e. an antioxidant, a flavoring, and a preservative). Those of skill in the art will understand that a single ingredient may have more than one function. For example, one element of a formulation may be both an antioxidant and a preservative or flavoring, or serve some other desired function in a formulation. Preferred antioxidants include butylated hydroxytoluene (BHT), butylated hydroxyanisole (BHA), and combinations thereof. Suitable preservatives comprise methylparaben, methylparaben sodium, propylparaben, and combinations thereof. Propylparaben and methylparaben are preferred preservatives, and combinations of the two are more preferred.

Flavorings suitable to include in liquid solutions of the invention for humans are fruit syrups such as grape syrup, grape cherry syrup, orange syrup, and cherry syrup, bubble gum, almond oil, anise oil, clove oil, lemon oil, licorice fluid extract, orange oil, peppermint oil, other mint oils, vanilla tincture, and various combinations thereof. Preferred flavorings include grape syrup, cherry syrup, and bubble gum. For animals, these flavorings also may be used where appropriate. Other flavors suitable for inclusion in formulations for animals are known in the art. For example, U.S. Pat. No. 3,645,753 describes a meat flavoring composition. Those of skill in the art will recognize that one, two, three, or even more flavorings may be combined in a formulation to yield a desired flavor. For example, bubble gum, grape, and cherry flavorings may be combined in a single formulation.

Other elements that optionally may be included in formulations of the invention include amino acids, vitamins, minerals, phospholipids, cyclodextrins, triglycerides, diglycerides, monoglycerides, ionic surfactants, non-ionic surfactants, bile salts, fatty acids, sweeteners, buffers, or any combinations thereof. Those of skill in the art will recognize that the inclusion of such additional elements in any particular formulation is dependent, at least in part, upon the individuals to whom the formulation is to be administered. For example, flavoring along with a sweetener may be particularly desirable in formulations for children or dogs, but less desirable in those formulations for adolescents or cats. Similarly, those of skill in the art will recognize that the specific disease(s) being treated or objective(s) of treatment may effect the addition of elements to formulations.

Phospholipids suitable for inclusion in formulations of the invention include phosphotidyl choline, phophotidyl ethanolamine, sphingomylein, lauroyl polyoxylglyceride, linoleoyl polyoxylglyceride, oleoyl polyoxylglyceride, lecithin, soy isoflavones, and combinations thereof.

Cyclodextrins suitable for inclusion in liquid solutions include α cyclodextrin, β cyclodextrin, δ cyclodextrin, γ cyclodextrin, and combinations thereof.

Suitable triglycerides that may be included in formulations of the invention are olive oil, safflower oil, soybean oil, sunflower oil, or combinations thereof. A suitable bile salt, i.e. bile acid, to include in the invention is cholesterol, or derivatives thereof.

Fatty acids that may be included in the invention are oleic acid, stearic acid, α-lipoic acid, ethyl oleate, myristic acid, palmitic acid, sorbitan monolaurate, sorbitan monooleate, sorbitan monopalmitate, sorbitan monostearate, sorbitan trioleate, and combinations thereof.

Any of the following amino acids may be included in formulations of the invention: alanine, arginine, aspartic acid, choline, folic acid, glutamine, glycine, histidine, isoleucine, leucine, lysine, methionine, phenylalanine, serine, threonine, tryptophan, tyrosine, valine, and combinations thereof.

Formulations of the invention may include a variety of sweeteners such as aspartame, calcium saccharate, dextrose, fructose, maltodextrin, maltose, mannitol, polydextrose, potassium sorbate, saccharin, saccharin calcium, saccharin sodium, sorbitol, sucralose, sucrose, sugar, xanthane gum, xylitol, xylose, and combinations thereof. Preferred sweeteners include saccharin sodium, sorbitol, and combinations of saccharin sodium and sorbitol.

Buffers may be included in formulations of the invention. In particular, any of the following may be included: ascorbic acid, ascorbyl palmitate, calcium sulfate, citric acid, dibasic sodium phosphate, monobasic sodium phosphate, potassium carbonate, potassium citrate, sodium acetate, sodium ascorbate, sodium bicarbonate, sodium carbonate, sodium citrate, sodium lauryl sulfate, sodium metabisulfate, and combinations thereof.

Surfactants suitable for inclusion in formulations are aqueous sodium laurel sulfate, a tocopherol excipient, tocopherol polyethyleneglycol, beta-carotene, lycopene, and combinations thereof.

It will be clear to the skilled artisan that a variety of optional ingredients may be included in formulations of the invention. Exemplary concentration ranges for various ingredients include: 1-90% PEG 400 (w/w), 1-90% propylene glycol, 1-90% glycerine, 1-75% chremophor EL, 1-75% labrasol (i.e. caprylocarproyl polyoxyglycerides), 1-75% Captex 355 (caprylic and capric acid triglycerides), 1-75% labrafil M 2125 CS (linoleoyl polyoxylglycerides), 1-75% Captex 500 P (glyceryl triacetate), 0.1-80% polysorbate 80, 0.1-80% 1-10% sodium lauryl sulfate in water, 0.01-15% methylparapben, 0.01-10% propylparaben, 0.1-25% sorbitol, 0.01-15% sodium benzoate, 0.01-5% sodium metabisulfate, 0.005-5% citric acid, 0-2% flavoring (e.g. bubble gum, grape, cherry), 0-2% saccharin sodium, 0-0.2% BHA, 0-0.02% BHT, and 0-85% water. Preferred concentration ranges for various ingredients include: 10-60% (w/w) PEG 400, 30-60% propylene glycol, 10-60% glycerine, 35% chremophor EL, 25-60% labrasol (i.e. caprylocarproyl polyoxyglycerides), 50-60% Captex 355 (caprylic and capric acid triglycerides), 60% labrafil M 2125 CS (linoleoyl polyoxylglycerides), 0.25% polysorbate 80, 0.2-1.0% methylparapben, 0.02-0.5% propylparaben, 11.25% sorbitol, 0.05-1.0% sodium benzoate, 0.1% sodium metabisulfate, 0.01-1.8% citric acid, 0.1-0.15% flavoring (e.g. bubble gum, grape, cherry), 1% saccharin sodium, 0.01% BHA, 0.01% BHT, and 0-64% water.

One exemplary embodiment of a liquid formulation of the invention comprises weight to weight (w/w) 0.2% simvastatin, 38.47% polyethylene glycol 400, USP, 0.2% methylparaben, NF, 0.02% propylparaben, NF, 0.01% butylated hydroxytoluene, NF, 60% glycerine, USP, 0.1% flavoring, and 1.0% saccharin sodium.

Another exemplary embodiment of the invention comprises liquid formulation comprising weight to weight (w/w) 0.2% simvastatin or simvastatin and atorvastatin, 38.22% polyethylene glycol, USP, 0.2% methylparaben, NF, 0.02% propylparaben, NF, 0.01% butylated hydroxytoluene, NF, 60% glycerine, USP, 0.1% a first flavoring, 0.15% a second flavoring, 0.1% a third flavoring, and 1.0% saccharin sodium. Preferred flavorings are grape, cherry, and bubble gum flavors.

Several preferred embodiments of formulations include, but are not limited to, the following: (1) 0.2% (w/w) simvastatin or simvastatin and atorvastatin, 59.8% polyethylene glycol (PEG) 400, 0.2% methylparaben, 0.02% propylparaben, 0.01% butylated hydroxyanisole (BHA), 10% sorbitol, 0.1% grape flavor, and 29.57% water; (2) 0.2% simvastatin or simvastatin and atorvastatin, 30% propylene glycol, 30% polyethylene glycol (PEG) 400, 0.2% methylparaben, 0.02% propylparaben, 0.01% butylated hydroxyanisole (BHA), 0.01% butylated hydroxytoluene (BHT), 39.46% glycerine, and 0.1% grape flavor; and (3) 0.2% simvastatin or simvastatin and atorvastatin, 59.8% polyethylene glycol (PEG) 400, 0.2% methylparaben, 0.02% propylparaben, 0.01% butylated hydroxytoluene (BHT), 10% glycerine, 10% sorbitol, 0.1% grape flavor, and 19.67% water.

The invention further includes methods of treating high cholesterol that comprise administering a liquid formulation of the invention to a subject orally. When treating a human, the individual is preferably a child or adolescent about 1-20 years old, more preferably 8-17 years old, has heterozygous familial hypercholesterolemia, and needs to lower levels of total cholesterol (total-C), low density lipoprotein cholesterol (LDL-C), and Apolipoprotein B (Apo B). It is preferred that the liquid solutions of statins are administered in conjunction with diet and lifestyle modifications. If administered to a human female, it is preferred that the female is at least one year post-menarche. When administered to an animal, it is expected that the animal is a companion animal such as a dog or cat. Other animals may also be treated with formulations of the invention where there is a desire to reduce the animal's total-C, LDL-C, or Apo B levels.

Ideally, dosages are monitored regularly using techniques well-known to the skilled artisan and adjusted as needed, preferably about every four weeks, to achieve the desired goal(s).

Herein, “individual” or “subject” refers to a human or an animal unless otherwise specified. Humans include children and adolescents. Animals include companion animals such as dogs and cats, as well as, e.g. ungulates, other mammals, birds, and fish.

“Statin” or “statins” is used herein to generally refer to a class of drugs that lower cholesterol levels by inhibiting the enzyme HMG-CoA reductase. Unless specified, it is understood that “statin” refers to both a single composition or a combination of compositions.

Unless defined otherwise, all technical and scientific terms used herein have the same meaning as is commonly understood by one of skill in the art to which this invention belongs at the time of filing. All patents and publications referred to herein are incorporated by reference herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION

Statins, in general, are delivered in solid form, e.g. tablet, because they tend to be difficult to solubilize and maintain in solution. The invention provides new formulations that provide stable, liquid solutions containing one or more statins, e.g. simvastatin, atorvastatin, etc., as the active ingredient(s). The solutions are suitable for oral ingestion as part of a treatment to reduce high cholesterol and particularly suitable to treat familial hypercholesterolemia, particularly heterozygous familial hypercholesterolemia (HeFH), in children and adolescents that are about 8-17 years old. Preferably the invention is used as an adjunct to diet and lifestyle modifications to reduce total cholesterol, low density lipoprotein cholesterol (LDL-C), and Apolipoprotein B (Apo B) levels in adolescent boys and girls who are about one year or more post-menarche, about 10-17 years old, and have HeFH or at high risk of developing HeFH. Formulations of the invention may also be used to treat similar conditions or diseases that occur in animals. It is expected that formulations of the invention will be most desirable for companion animals such as dogs or cats.

In all instances, formulations of the invention include a solubilizer that allows the statin(s) to enter and remain in solution. Previously, the statins in formulations of the invention were not available in stable, liquid solutions that would be suitable for oral ingestion and the treatment of high cholesterol. Herein are disclosed formulations, and methods of preparing such formulations, that include one or more solubilizers to yield stable liquid solutions that include a statin and are suitable for oral ingestion.

Potential solubilizers (a.k.a. vehicles) for oral solutions or suspensions include: propylene glycol, minerals, propylene glycolmonostearate, propylene glycol alginate, natural glycerine, niacin, synthetic glycerine, vitamins, sorbitol, alcohols, myristyl alcohol, carboxymethylcellulose, labrasol, copovidone, Captex 355, croscarmellose sodium, polyethylene glycol (PEG) 400, other PEGs (e.g. 200, 300, 1000, 1450, 1540, etc. and up to 10,000), crospovidone, ethyl cellulose, aqueous polysorbate 80, cellulose, other polysorbates (20, 40, 60), oxidized cellulose, polyoxyl 10 oleoyl ether, cellulose sodium phosphate, polyoxyl 20 cetostearyl, hyopromellose, poloyxyl 35 castor oil, polyoxyl 40 hydrogentated castor oil, polyoxyl 40 stearate, poloxyl lauryl ether, poloxyl oleate, and poloxyl stearyl ether.

Exemplary solutions of the invention include a statin, such as simvastatin, at least one vehicle to solubilize the statin, and optionally, one or more antioxidants, flavors, or preservatives. Vitamins, amino acids, minerals, phospholipids, cyclodextrins, triglycerides, diglycerides, monoglycerides, surfactants, bile salts, fatty acids, sweeteners, or buffers also may be included in solutions of the invention.

It is envisioned that formulations of the invention can include any statin that is not presently available in a liquid formulation that can be ingested orally and can be solubilized using one or more of the solublizers disclosed herein by using the methods disclosed herein. Combinations of statins may be used and include one or more Type I or Type II statins or combinations thereof. Suitable Type I statins include lovastatin, mevastatin, pravastatin, and simvastatin. Suitable Type II statins typically have a fluorophenyl group in place of the butyrl group that is present in Type I statins and include atorvastatin, cerivastatin, fluvastatin, rosuvastatin, and pitavastatin. Multiple forms of statins may also be used in the formulations. That is, a statin may be present as an acid (e.g. carboxylic acid), salt (including calcium, sodium, potassium, and magnesium salts), or neutral (closed lactone ring) form. Those of skill in the art will recognize that the desired combination(s) of statins will depend upon the end user, the objective(s) of the treatment, and the solubility of the combination.

Phospholipids suitable for inclusion in oral solutions or suspensions include: phosphotidyl choline, phophotidyl ethanolamine, sphingomylein, lauroyl polyoxylglyceride, linoleoyl polyoxylglyceride, oleoyl polyoxylglyceride, lecithin, and soy isoflavones.

Solutions of the invention may also include α, β, δ, and γ cyclodextrins. Triglycerides may also be included in solutions of the invention, such as: olive oil, safflower oil, soybean oil, sunflower oil, diglycerides, monoglycerides, and diacetylated monoglycerides. Bile salts, for example cholesterol, may be included in solutions of the invention.

Fatty acids suitable for inclusion in solutions of the invention include: oleic acid, stearic acid, α-lipoic acid, ethyl oleate, myristic acid, palmitic acid, sorbitan monolaurate, sorbitan monooleate, sorbitan monopalmitate, sorbitan monostearate, and sorbitan trioleate. Amino acids suitable for inclusion in solutions of the invention include: alanine, arginine, aspartic acid, choline, folic acid, glutamine, glycine, histidine, isoleucine, leucine, lysine, methionine, phenylalanine, serine, threonine, tryptophan, tyrosine, and valine.

Suitable sweeteners include: aspartame, calcium saccharate, dextrose, fructose, maltodextrin, maltose, mannitol, polydextrose, potassium sorbate, saccharin, saccharin calcium, saccharin sodium, sorbitol, sucralose, sucrose, sugar, xanthane gum, xylitol, and xylose. Suitable flavors include: grape syrup, grape cherry syrup, bubble gum, almond oil, anise oil, cherry syrup, clove oil, lemon oil, licorice fluid extract, orange oil or syrup, peppermint oil, and vanilla tincture.

Possible buffers that may be included in the liquid solutions are: ascorbic acid, ascorbyl palmitate, calcium sulfate, citric acid, dibasic sodium phosphate, monobasic sodium phosphate, potassium carbonate, potassium citrate, sodium acetate, sodium ascorbate, sodium bicarbonate, sodium carbonate, sodium citrate, sodium lauryl sulfate, and sodium metabisulfate.

Preservatives that may be included in the invention are methylparaben, and methylparaben sodium. Antioxidants that may be included in the invention are butylated hydroxytoluene, and butylated hydroxyanisole.

Any of the following ionic or non-ionic surfactants may be included in oral solutions or suspensions of the invention: sodium laurel sulfate in water, tocopherols excipient, tocopherol polyethyleneglycol, beta-carotene, and lycopene.

Exemplary formulations are comprised of percent weight to weight (% w/w) of 0.05-10% simvastatin, and any of the following: 5-75% PEG 400, 5-75% propylene glycol, 5-75% glycerine, 5-60% chremophor EL, 5-60% labrasol (i.e. caprylocarproyl polyoxyglycerides), 5-60% Captex 355 (caprylic and capric acid triglycerides), 5-60% labrafil M 2125 CS (linoleoyl polyoxylglycerides), 5-60% Captex 500 P (glyceryl triacetate), 0.1-60% polysorbate 80, 0.1-60% 5% sodium lauryl sulfate in water, 0.01-1.5% methylparapben, 0.01-1.0% propylparaben, 1-15% sorbitol, 0.01-1.5% sodium benzoate, 0.01-0.5% sodium metabisulfate, 0.005-2.5% citric acid, 0-0.5% flavoring (e.g. bubble gum, grape, cherry), 0-2% saccharin sodium, 0-0.02% BHA, 0-0.02% BHT, and 0-75% water.

Preferred formulations are comprised of percent weight to weight (% w/w) of 0.2-0.25% of a statin, preferably simvastatin, and any of the following: 10-60% PEG 400, 30-60% propylene glycol, 10-60% glycerine, 35% chremophor EL, 25-60% labrasol (i.e. caprylocarproyl polyoxyglycerides), 50-60% Captex 355 (caprylic and capric acid triglycerides), 60% labrafil M 2125 CS (linoleoyl polyoxylglycerides), 0.25% polysorbate 80, 0.2-1.0% methylparapben, 0.02-0.5% propylparaben, 11.25% sorbitol, 0.05-1.0% sodium benzoate, 0.1% sodium metabisulfate, 0.01-1.8% citric acid, 0.1-0.15% flavoring (e.g. bubble gum, grape, cherry), 1% saccharin sodium, 0.01% BHA, 0.01% BHT, and 0-64% water.

Two exemplary formulations are as follows: (1) 0.2% simvastatin, 38.47% polyethylene glycol 400, USP, 0.2% methylparaben, NF, 0.02% propylparaben, NF, 0.01% butylated hydroxytoluene, NF, 60% glycerine, USP, 0.1% grape flavor, and 1.0% saccharin sodium; and (2) 0.2% simvastatin, 38.22% polyethylene glycol, USP, 0.2% methylparaben, NF, 0.02% propylparaben, NF, 0.01% butylated hydroxytoluene, NF, 60% glycerine, USP, 0.1% grape flavor, 0.15% bubble gum flavor, 0.1% cherry flavor, and 1.0% saccharin sodium.

### Type I Statins

Simvastatin

The chemical name of simvastatin is butanoic acid, 2,2-dimethyl-,1,2,3,7,8,8a-hexahydro-3,7-dimethyl-8-[2-(tetrahydro-4-hydroxy-6-oxo-2H-pyran-2-yl)-ethyl]-1-naphthalenyl ester, [1S-[1α,3α,7β,8β(2S*,4S*),-8αβ]]. Its molecular formula is C₂₅H₂₈O₅, and its structural formula is:

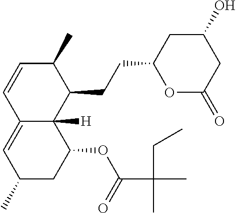

Simvastatin has a molecular mass of 418.57. It is a lactone that is readily hydrolyzed in vivo to the corresponding β-hydroxyacid, a potent inhibitor of 3-hydroxy-3-methylglutaryl coenzyme A (HMG-CoA) reductase.

In solid form, simvastatin is a white to off-white, nonhygroscopic, crystalline powder that is nearly insoluble in water and freely soluble in chloroform, methanol, and ethanol. Due to its poor solubility in water, the toxicity of chloroform, methanol, and ethanol, and short shelf-life in liquid form, liquid formulations of simvastatin suitable for treating humans, especially children and adolescents, have not been previously available. The invention provides safe, stable, liquid formulations of simvastatin particularly suited for treating children and adolescents.

Both simvastatin and its β-hydroxyacid metabolite are highly bound (approximately 95-98%) to human plasma proteins. Rat studies indicate that when radiolabeled simvastatin was administered, simvastatin-derived radioactivity crossed the blood-brain barrier. Since simvastatin undergoes extensive first-pass extraction in the liver, the availability of the drug to the general circulation is low (<5%). The major active metabolites of simvastatin present in human plasma are the 0 hydroxyacid of simvastatin and its 6′-hydroxy, 6′-hydroxymethyl, and 6′-exomethylene derivatives.

Following an oral dose of ¹⁴C-labeled simvastatin in man, 13% of the dose was excreted in urine and 60% in feces. Plasma concentrations of total radioactivity (simvastatin plus ¹⁴C-metabolites) peaked at 4 hours and declined rapidly to about 10% of peak by 12 hours post-dose. Peak plasma concentrations of both active and total inhibitors were attained within 1.3 to 2.4 hours post dose. While the recommended therapeutic dose range in an adult is 5 mg/day to 80 mg/day, there was no substantial deviation from linearity of area under the curve (AUC) of inhibitors in the general circulation with an increase in dose to as high as 120 mg. Relative to the fasting state, the plasma profile of inhibitors was not affected when simvastatin was administered immediately before an American Heart Association (AHA) recommended low-fat meal.

Lovastatin

Lovastatin, also known as (1S,3R,7S,8S,8aR)-8-{2-[(2R,4R)-4-hydroxy-6-oxooxan-2-yl]ethyl}-3,7-dimethyl-1,2,3,7,8,8a-hexahydronaphthalen-1-yl (2S)-2-methylbutanoate or mevinolin, has the molecular formula 404.54 g/mol. Its chemical formula is C₂₄H₃₆O₅ and its chemical structure is as follows:

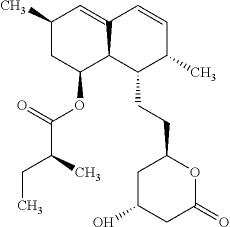

In its solid form, lovastatin is a white powder. Like simvastatin and other type I statins, both acid and salt forms of lovastatin may occur and be present in liquid formulations of the invention.

Mevastatin

Mevastatin, or (1S,7S,8S,8aR)-8-{2-[(2R,4R)-4-hydroxy-6-oxotetrahydro-2H-pyran-2-yl]ethyl}-7-methyl-1,2,3,7,8,8a-hexahydronaphthalen-1-yl (2S)-2-methylbutanoate, has a molecular weight of 408.534 g/mol, chemical formula of C₂₃H₃₄O₅, and the following structural formula:

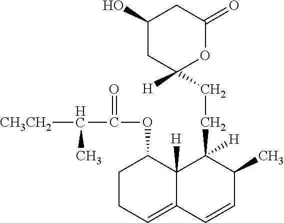

Pravastatin

In its sodium salt form pravastatin has the following structural formula:

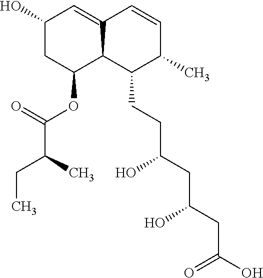

Pravastatin has the following chemical formula (bR,dR,1S,2S,6S,8S,8aR)-1,2,6,7,8,8a-Hexahydro-b,d,6-trihydroxy-2-methyl-8-[(2S)-2-methyl-1-oxobutoxy]-1-napht haleneheptanoic acid monosodium salt, and a molecular weight of 446.51.

### Type II Statins

Atorvastatin

Atorvastatin has the chemical formula (3R,5R)-7-[2-(4-fluorophenyl)-3-phenyl-4-(phenylcarbamoyl)-5-(propan-2-yl)-1H-pyrrol-1-yl]-3,5-dihydroxyheptanoic acid. Its molecular weight is 558.64, and chemical formula is C₃₃H₃₅FN₂O₅. Its structural formula is:

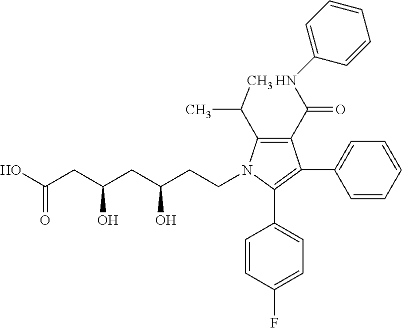

In its calcium salt form atorvastatin is known as Lipitor®. Atorvastatin calcium is a white to off-white crystalline powder that is insoluble in aqueous solutions of pH 4 and below and is very slightly soluble in distilled water, pH 7.4 phosphate buffer, and acetonitrile, slightly soluble in ethanol, and freely soluble in methanol. The empirical formula of atorvastatin calcium is (C₃₃H₃₄FN₂O₅)₂Ca.3H₂O and its molecular weight is 1209.42. Its structural formula is:

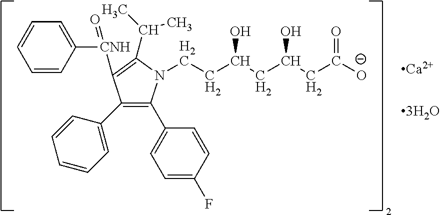

Cerivastatin

Cervistatin, also known as (3R,5S)-7-[4-(4-fluorophenyl)-5-(methoxymethyl)-2,6-dipropan-2-yl-pyridin-3-yl]-3,5-dihydroxy-hept-6-enoic acid, has a molecular weight of C₂₆H₃₄FNO₅. It is a synthetic member of the class of statins. Cerivastatin sodium is sodium[S—[R*,S-(E)]-7-[4-(4-b fluorophenyl)-5-methoxymethyl)-2,6bis(1-met ylethyl) 3-pyridinyl]-3,5-dihydroxy+heptenoate. The empirical formula for cerivastatin sodium is C₂₆H₃₃FN0₅Na and its molecular weight is 481.5. Cerivastatin sodium is a white to off-white hygroscopic amorphous powder that is soluble in water, methanol, and ethanol, and very slightly soluble in acetone. Its structural formula is:

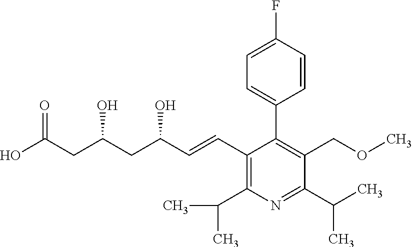

Fluvastatin

Fluvastatin sodium is [R*,S*-(E)]-(±)-7-[3-(4-fluorophenyl)-1-(1-methylethyl)-1H-indol-2-yl]-3,5-dihydroxy-6-heptenoic acid, monosodium salt. The empirical formula of fluvastatin sodium is C₂₄H₂₅FNO4.Na, its molecular weight is 433.46. Its structural formula is:

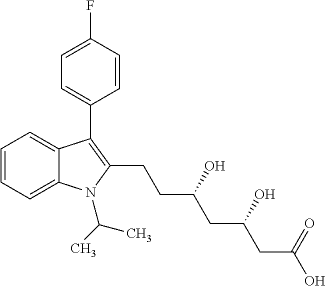

Rosuvastatin

The chemical name of rosuvastatin is bis[(E)-7-[4-(4-fluorophenyl)-6-isopropyl-2-[methyl(methylsulfonyl)amino]pyrimidin-5-yl](3R,5S)-3,5-dihydroxyhept-6-enoic acid]calcium salt. Its structural formula is

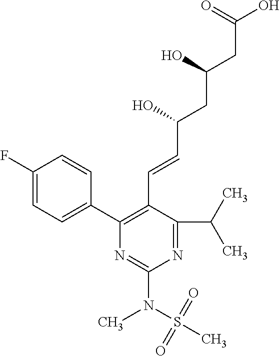

Rosuvastatin calcium is known as Crestor®. The empirical formula for rosuvastatin calcium is (C₂₂H₂₇FN₃O₆S)₂Ca and its molecular weight is 1001.14. Rosuvastatin calcium is a white amorphous powder that is sparingly soluble in water and methanol, and slightly soluble in ethanol. Rosuvastatin calcium is a hydrophilic compound with a partition coefficient (octanol/water) of 0.13 at pH of 7.0.

Pitavastatin

Pitavastatin, or (3R,5S,6E)-7-[2-cyclopropyl-4-(4-fluorophenyl)quinolin-3-yl]-3,5-dihydroxyhept-6-enoic acid, has the molecular weight of 421.461 and chemical formula of C₂₅H₂₄FNO₄. It is usually found as a calcium salt.

### Safety of Statins

Reduction of LDL by statin therapy is primarily due to hepatic inhibition of cholesterol synthesis leading to an upregulation of LDL receptors which finally enhances the clearance of LDL from the serum. Statins also have a direct impact on the vascular system, which is potentially relevant for prevention of atherosclerotic complications. Some of these effects are mediated by isoprenoid intermediates involved in cholesterol biosynthesis, which regulates cellular distribution and function of small GTPases. Therefore, the inhibition of cholesterol synthesis with statin therapy may have an effect on adrenal and gonadal steroidogenesis because hormone synthesis requires an efficient intracellular pool of free cholesterol. A possible impairment of steroidogenesis could be due to the direct inhibition of cholesterol synthesis or could be caused by a reduction of LDL-particle uptake by steroidogenesis tissues.

A number of studies have assessed the impact of HMG-CoA reductase inhibitors on steroidogenesis. In vivo, dogs treated with very high doses of lovastatin showed a reduction in testicular endocrine function. In addition, simvastatin (20 mg/kg per day) lowered stimulated progesterone levels in rabbits with defective LDL-receptors. However, in humans given up to 80 mg/day lovastatin, no effect on adrenal and gonadal steroidogenesis was demonstrated under basal conditions. This is further supported by studies demonstrating no clear adverse effect on basal gonadotropin and sex hormone serum levels in hypercholesterolemic patients given statins.

A study by Travia et al., J Clin Endocrinol Metab 1995; 80(3): 836-840, explored maximally stimulated adrenocortical and testicular steroidogenesis in males (age 18-50 years) treated for long periods with either simvastatin or pravastatin. The results of this study indicated that even during maximum stimulation, HMG-CoA reductase inhibitor therapy did not interfere with adrenal and testicular steroidogenesis. Serum testosterone levels remained unchanged and all urinary metabolite levels were very similar to those measured prior to therapy. The authors concluded that these data suggest that treatment with a HMG-CoA reductase inhibitor does not have any detrimental effect on steroidogenesis in patients with HeFH.

In adults and children, muscle and liver toxicity caused by statin therapy constitutes the main concern in clinical practice. In a systematic safety review in adults it was estimated that a very rare occurrence of rhabdomyolysis of 3 per 100 000 person-years was associated with atorvastatin, simvastatin, lovastatin, pravastatin, and fluvastatin. In the systematic review conducted by Avis et al., Arterioscler Thromb Vasc Biol 2007; 27(8):1803-1810, the total number of subjects or person-years was far too small to estimate the risk of rhabdomyolysis.

### Statin Oral Solutions

Statin oral solutions of the invention include about 0.1-25 mg/ml total statin. Preferred statin solutions include 2-5 mg/ml total statin. Exemplary simvastatin oral solutions includes about 2 mg/ml simvastatin. Statin oral solutions are indicated as an adjunctive therapy to diet to reduce the risk of total mortality by reducing coronary heart disease (CHD) deaths and to reduce the risk of non-fatal myocardial infarction, stroke, and the need for revascularization procedures in patients at high risk of coronary events such as atherosclerotic cardiovascular disease; to reduce elevated total-cholesterol (total-C), LDL-C, apolipoprotein B (Apo B), triglycerides (TG) and increase high-density lipoprotein cholesterol (HDL-C) in patients with primary hyperlipidemia (heterozygous familial and nonfamilial) and mixed dyslipidemia; to reduce elevated TG in patients with hypertriglyceridemia and reduce TG and very low-density lipoprotein cholesterol (VLDL-C) in patients with primary dysbetalipoproteinemia; to reduce total-C and LDL-C in adult patients with homozygous familial hypercholesterolemia; and to reduce elevated total-C, LDL-C, and Apo B in boys and girls, 8 to 17 years of age with heterozygous familial hypercholesterolemia (HeFH) after failing an adequate trial of diet therapy.

### Dosage and Administration

It is expected that statin oral formulations will be used as an adjunct to diet to reduce total-C, LDL-C, and Apo B levels in adolescent boys and girls who are at least one year post-menarche, i.e. about 10-17 years old, with HeFH, if after an adequate trial of diet therapy the following conditions exist: LDL-C remains ≧190 mg/dl, or LDL-C remains ≧160 mg/dl, and there is a familial history of premature cardiovascular disease or two or more other risk factors for cardiovascular disease are present in the patient. Statin oral formulations also will be expected to be used in patients having homozygous familial hypercholesterolemia as an adjunct to other lipid lowering treatments (e.g. LDL apheresis) or alone if such other therapies are not available.

In adults the dosage of statin may range from 1-120 mg/day, the preferred dosage is 5-80 mg/day. It is usually recommended that the starting dose is 20-40 mg once a day in the evening. For adult patients at high risk of CHD, the recommended dose is 40 mg/day.

For children and adolescents, dosing is usually more conservative compared to adults so that the potential for dose-related adverse events is reduced. The minimum goal of treatment in pediatric and adolescent patients is to achieve a mean LDL-C<130 mg/dl. In adolescents (about 10-17 years old) with HeFH, the usual starting dose is 10 mg/day in the evening. The maximum preferred dosage for an adolescent is 40 mg/day with doses ranging from 10-80 mg/day. For children (about 8-9 years old) with HeFH, the usual starting dose is 5 mg/day in the evening, and the recommended dosage range is 5-20 mg/day in the evening. The maximum preferred dose for children about 8-9 years old with HeFH is 20 mg/day with doses ranging from 5-80 mg/day. In general, adjustments should be made at intervals of about 4 weeks or longer, but doses may be individualized and adjusted at any time as needed to achieve the desired therapeutic goal.

For patients having homozygous familial hypercholesterolemia, the recommended dosage is about 40 mg/day in the evening or 80 mg/day in three divided doses of 20 mg, and an evening dose of 40 mg. Dosages may individualized and even increased to achieve the desired therapeutic goal. In such instances, careful monitoring for potential adverse effects should be done using techniques well-known to the skilled artisan.

It is expected that for most children and adolescents, the preferred or recommended dosages will be used; however, doses may be adjusted to meet individual needs. Thus, a child or adolescent may receive the equivalent of 0.5-120 mg/day of statin, e.g. simvastatin, using the oral formulation, preferably the adolescent receives the equivalent of 5-40 mg/day of statin using the oral formulation and the child receives the equivalent of 5-20 mg/day of statin using the oral formulation. The liquid formulation may be given as a single dose, preferably in the evening, or in multiple doses. It may be given daily, preferred, or at multiple day intervals. An advantage of using the liquid formulation is the dosage may be more precisely calculated and provided. For example, a child or adolescent may receive the equivalent of 0.5, 1, 1.5, 2, 2.5, 3, 3.5, 4, 4.5, 5, 5.5, 6, 6.5, 7, 7.5, 8, 8.5, 9, 9.5, 10, 10.5, . . . 16, 16.5, 17, 17.5, 18, 18.5, 19, 19.5, 20, 20.5, . . . 36, 36.5, 37, 37.5 38, 38.5, 39, 39.5, 40, 40.5, . . . 78, 78.5, 79, 79.5, 80, 80.5 . . . 118, 118.5, 119, 119.5, or even 120 mg/day of statin using the liquid formulation. Single doses will include 0.5 to 25 mg/ml of statin, preferred doses will include 2-5 mg/ml statin.

In animals, the dosage will depend, in part, upon the species being treated, its age, and goal of treatment.

The specific dose and dosage regimen used will depend upon the individual being treated. It is recommended that total-C and LDL-C level be monitored regularly and the dose and dosing regimen adjusted as needed to achieve the desired effects. Factors that may effect the effectiveness of a particular dosage include, but are not limited to, age, gender, body surface area, renal function, and pubertal development stage. Other factors include medications that inhibit the cytochrome P450 enzymes, especially CYP3A4 and CYP2C9; medications that increase the serum concentration of statins such as macrolide antibiotics, antifungal agents, HIV-protease inhibitors, calcium channel blockers, and cyclosporine; and enzyme inducers (e.g., rifampin, barbiturates, and carbamazepine) that decrease statin serum concentrations.

The optimal age at which to initial lipid-lowering therapy to decrease the risk of symptomatic adulthood coronary artery disease (CAD) has not been determined. It is preferred that a statin is not used to treat children less than age 8 and girls that are less than one year post-menarche; however, due to evidence that macrovascular and atherosclerotic cardiovascular diseases begin in childhood, it is envisioned that relatively low doses of a statin, such as simvastatin, may be used in children less than age 8 or in girls that are less than one year post-menarche. In such instances, the liquid formulation will be particularly useful as very low doses, e.g. 0.5-5.0 mg/day, may be easily calculated and administered.

### Use of Statin in Tablet (Solid) Form in Children and Adolescents

Ducobu et al., Simvastatin use in children, Lancet 1992; 339(8807):1488, incorporated herein by reference, performed a study of simvastatin in 32 children (22 male; 10 female) younger than 17 years of age with hyperlipidemia. Male and female patients were allowed to enter this protocol if their total cholesterol was above 300 mg/dl after diet therapy for six months. Children under the age of 10 started at an initial dose of 5 mg once daily. Titration to 10 mg daily after 4 weeks and to 20 mg after another 4 weeks was possible. Older children began on a daily dose of 10 mg, which was increased to 20 mg after 6 weeks and, after an additional 6 weeks, to 40 mg if necessary. The mean dosage was 16 mg once daily (37% received 10 mg, 38% 20 mg, and 25% 40 mg). The concentration of circulating LDL-C at week 104 had fallen by 40-60% in 7 patients, by 20-40% in 8 patients, and by 15-20% in 1 patient. Few adverse effects were noted over a follow-up period of at least 24 months and growth and development remained normal.

Dirisamer et al., The effect of low-dose simvastatin in children with familial hypercholesterolaemia: a 1-year observation. Eur J Pediatr 2003; 162(6): 421-425, incorporated herein by reference, conducted a single-center, open-labeled and diet-controlled, 18-month study to investigate the efficacy and safety of low-dose simvastatin on lipids and lipoprotein in children with HeFH with the aim to reduce LDL-C and total cholesterol concentrations to moderate levels with the lowest possible dosage. The group of 20 girls and boys aged 10-17 years (13.0±2.4 years), with HeFH (i.e., LDL-C>190 mg/dl) underwent a 3-month cholesterol-lowering diet prior to receiving 12 months of simvastatin therapy. The simvastatin dosage at the beginning of the trial was 5 mg for patients with LDL-C concentrations <220 mg/dl, and 10 mg simvastatin for those with LDL-C concentrations >200 mg/dl. If the range of LDL-C of 150-170 mg/dl was not reached within the first 8 weeks, the daily dosage was increased stepwise up to 20 mg. The cut-off level for LDL-C<170 mg/dl (range 150-170 mg/dl) was determined according to the high mean concentrations of LDL-C of the study population. A total of nine patients started with 5 mg simvastatin and in five of these patients the daily dosage was increased to 10 mg after the first visit. The other four patients reached the recommended therapeutic level (LDL-C<170 mg/dl) within the first period with 5 mg simvastatin daily.

A total of 11 patients started with 10 mg simvastatin and in five of these patients the daily dosage was increased up to 20 mg simvastatin after the first visit. Six patients reached the recommended therapeutic level within the first period with 10 mg simvastatin daily. The percentage decrease in LDL-C concentration was 25% (p<0.001) in the 5 mg simvastatin period, 30% (p<0.0001) in the 10 mg simvastatin period, and 36% (p<0.001) in the 20 mg simvastatin period. The percentage decrease in total cholesterol was 19% (p<0.001) in the 5 mg simvastatin period, 26% (p<0.0001) in the 10 mg simvastatin period, and 29% (p<0.01) in the 20 mg simvastatin period.

Side effects, that could be related to simvastatin were few and equally distributed among the three dosage periods and determined to be of no clinical relevance. Most disappeared after a couple of days. There were only three subjects with abnormal levels of routine safety parameters. Two patients (one receiving 5 mg/day and the other receiving 10 mg/day) showed slightly higher values of creatine kinase (CK) and one (10 mg/day) expressed transiently elevated concentrations of alanine transamine (ALAT) (GPT) and GGT. The other subjects did not show any changes in routine parameters. No dose relationship was found.

Dirisamer et al. found simvastatin to be an effective and safe medical therapy in children and adolescents with HeFH. In ten patients, the starting dosage did not need to be increased. A percentage decrease of 25% and 30% of LDL-C in the 5 mg and 10 mg simvastatin period seemed to be more effective compared to other statins tested in adolescent boys and girls. This is an almost 10% higher reduction compared with other statins tested in young HeFH patients. Knipscheer et al., Pediatr Res 1996; 39(5):867-871, showed an average decrease in LDL-C concentration of 23% in HeFH children and adolescents receiving 5 mg and 10 mg pravastatin, another statin. Lambert et al., Pediatrics 1996; 97(5):619-628, found an LDL-C reduction of 21% and 24% in children and adolescents receiving 10 mg and 20 mg lovastatin, respectively. It has been reported that a significant response was observed at the lowest dose of lovastatin used (10 mg/day) and this response accounted for more than 50% of the mean reduction in the LDL-C level observed with the highest dose of 40 mg/dl. This effect was also observed in this simvastatin trial where the lowest dose of simvastatin used (5 mg/day) accounted for more than 70% of the mean reduction in the LDL-C level with the highest dose of 20 mg/day used.

An international, multicenter (n=9), double-blind, randomized, parallel study of 173 pediatric HeFH patients with Zocor®. Entry criteria included children aged 10 to 17 years with LDL-C levels between 4.1 and 10.3 mmol/L and 1 parent with a confirmed diagnosis of HeFH. Boys were in Tanner stage 11 or above, and girls were postmenarchal for at least 1 year before the initiation of the study. After a 4-week diet/placebo run-in period, children were randomized to active treatment or matching placebo in a ratio of 3:2 and stratified by sex. Simvastatin was started at 10 mg/d and was increased at 8-week intervals to 20 and then 40 mg/d for the remainder of the study (period 1) and for the 24-week extension (period 2). Visits occurred every 4 weeks. The menstrual cycle was monitored throughout the study period by recording the first day of the menstrual flow. Tanner staging based on testicle size (boys) and breast size (girls) was used for pubertal development. Efficacy measurements (total-C, triglycerides, LDL-C, and HDL-C) and safety measurements (alanine transaminase (ALT), aspartate aminotransferase (AST), and creatine kinase (CK)) were performed at every visit or every other visit (Apolipoprotein B (ApoB) and Apolipoprotein A1 (ApoA1)).

A total of 175 children were included in the study: 69 were randomized to placebo, and 106 were randomized to simvastatin. Two children in the placebo group were excluded from the intention-to-treat (ITT) analysis because of loss of follow-up and withdrawal of consent. The majority of randomized children were boys (52% of the placebo group, 59% of the simvastatin group). In the 24-week extension, 144 patients elected to continue therapy and received simvastatin 40 mg or placebo.

Mean percent changes from baseline for lipids and lipoproteins at 24 weeks are shown in Table 1. Compared with placebo, simvastatin produced significant (P<0.001) reductions in total-C, LDL-C and ApoB Results from the extension at 48 weeks were comparable to those observed in the base study. Significant reductions in triglycerides were seen at weeks 8, 16, and 48. HDL-C and ApoA1 were increased for all weeks; however, these increases were only significant relative to placebo at week 24 (P<0.05).

TABLE 1
Lipid Lowering Effects of Simvastatin in Adolescent Patients
with HeFH (Mean Percent Change from Baseline).

| Dosage | | Total-C | | LDL-C | | HDL-C | | TG^{†} | | ApoB | |
|---|---|---|---|---|---|---|---|---|---|---|---|
| Placebo | % Change | 1.6 | (−2.2, 5.3) | 1.1 | (−3.4, 5.5) | 3.6 | (−0.7, 8.0) | −3.2 | (−11.8, 5.4) | −0.5 | (−4.7, 3.6) |
| | from Baseline | | | | | | | | | | |
| | (95% CI) | | | | | | | | | | |
| | Mean Baseline, | 278.6 | (51.8) | 211.9 | (49.0) | 46.9 | (11.9) | 90.0 | (50.7) | 186.3 | (38.1) |
| | mg/dL | | | | | | | | | | |
| | (SD) | | | | | | | | | | |
| Zocor ® | % Change | −26.5 | (−29.6, −23.3) | −36.8 | (−40.5, −33.0) | 8.3 | (4.6, 11.9) | −7.9 | (−15.8, 0.0) | −32.4 | (−35.9, −29.0) |
| | from Baseline | | | | | | | | | | |
| | (95% CI) | | | | | | | | | | |
| | Mean Baseline, | 270.2 | (44.0) | 203.8 | (41.5) | 47.7 | (9.0) | 78.3 | (46.0) | 179.9 | (33.8) |
| | mg/dL | | | | | | | | | | |
| | (SD) | | | | | | | | | | |

It is expected that the statin liquid formulations of the present invention will yield results comparable to those of Dirisamer et al., Ducobu et al., and with Zocor® described above when used in children or adolescents. In addition, because the dosing with the liquid formulations can be administered more precisely, it is expected that it will be easier to use very low doses in children and adolescents as compared to the solid form (tablet) of simvastatin.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Simvastatin Solubility Studies

A solubility study was performed using Simvastatin in various vehicles at a concentration of 2.5 mg/gram. The study was performed by visual observation of the samples at various time points followed by chemical testing of the centrifuged supernatant. Ten vehicles chosen for the solubility screen are referenced in Table 2 below.

TABLE 2
Vehicles Chosen for Solubility Screening

| Trade Name [Chemical Name] | Function(s) |
|---|---|
| Propylene Glycol, USP | Hydrophilic (H) |
| | Solvent/Preservative |
| Glycerine, USP | Hydrophilic (H) Solvent |
| Labrasol, USP/NF | Lipophilic (L)-Surfactant, Medium |
| [Caprylocaproyl Polyoxyglycerides] | Chain Triglyceride (MCT)/Solvent |
| Captex ® 355, NF | Medium Chain Triglyceride |
| [Triglycerides of Caprylic/ | (MCT)/Solvent |
| Capric Acid] | |
| E. pure Water | Solvent |
| Labrafil M 2125 CS, USP/NF | Lipophilic (L)-Surfactant, Medium |
| [Linoleoyl Polyoxylglycerides] | Chain Triglyceride (MCT)/Solvent |
| Captex ® 500 P, USP | Hydrophilic (H)-Solvent |
| [Glyceryl Triacetate] | |
| PEG 400, NF | Hydrophilic (H)-Solvent |
| [Polyethylene Glycol 400] | |
| Polysorbate 80, NF | Nonionic Surfactant |
| [Polyoxyethylene 20 Sorbitan | |
| Monooleate; Crillet 4 HP] | |
| 5% Sodium Lauryl Sulfate, NF | Anionic Surfactant/Wettng Agent |
| in Water | |

API Lot: Simvastatin (micronized), USP, Lupin Limited, Batch 080440008, Exp. July 2010

### Solubility Screening Process

One (1 g) of each vehicle was dispensed into an Eppendorf tube. Simvastatin, in the amount of 2.5 mg, was dispensed and transferred to each tube containing 1 gram of vehicle. (Due to the weighing accuracy of the balance per USP, 2.5 mg of simvastatin was dispensed for each sample in 1 gram of the vehicle.) Each tube was vortexed for about 20 seconds, and the tubes were transferred to a thermomixer for mixing at 1400 RPMs at 23° C. Mixing was stopped for visual observation of the samples at 30 minutes, 1 hour, 2 hours, 4 hours, and at about 21 hours. See Table 3 for results.

TABLE 3
Visual Observation of 2.5 mg Simvastatin in 1 g of Vehicle.

| Sample | Visual Appearance | | | | | |
|---|---|---|---|---|---|---|
| Identity | Initial (T = 0) | 30 Min | 1 Hr | 2 Hrs | 4 Hrs | ~21 Hrs |

| Propylene | Clear colorless | Clear colorless liquid | | |
|---|---|---|---|---|
| Glycol | liquid w/ white | | | |
| | powder | | | |
| Glycerine | Stiff clear, colorless liquid w/ white powder | | | |
| Labrasol | Pale yellow liquid | Pale yellow liquid | | |
| | w/ white powder | | | |
| Captex 355 | Clear colorless | Clear colorless | Clear colorless liquid w/ | Clear |
| | liquid w/ white | liquid w/ white | less white powder | colorless |
| | powder | powder | | liquid |

| | | |
|---|---|---|
| E. Pure | Partially cloudy | Cloudy liquid w/ white powder |
| Water | liquid w/ white | |
| | powder | |

| | | | |
|---|---|---|---|
| Labrafil M | Yellow liquid w/ white powder | Yellow liquid w/ | Clear |
| 2125 CS | | less white | yellow |
| | | powder | liquid |

| Captex 500 P | Clear colorless | Clear colorless liquid |
|---|---|---|
| | liquid w/ white | |
| | powder | |
| PEG 400 | Clear colorless | Clear colorless liquid |
| | liquid w/ white | |
| | powder | |
| Polysorbate | Yellow liquid w/ white powder | |
| 80 | | |
| 5% SLS in | Clear colorless | Clear colorless liquid w/ air bubbles |
| Water | liquid w/ white | |
| | powder, air bubbles | |

Samples not visually soluble, after about 21 hours, were heated to 60° C. while being mixed for 1 hour. Samples were allowed to reach room temperature, and visual observations were recorded. See Table 4. All samples were centrifuged for 10 minutes at 10,000 RPM.

TABLE 4
Visual Observation after Application of Heat (60° C.) for 1 Hour and
Cooled to Room Temperature.

| Sample Identity | Visual Appearance |
|---|---|
| Glycerine | Slightly hazy liquid w/ some white powder |
| E. Pure Water | Hazy liquid w/ white powder and air bubbles |
| Polysorbate 80 | Clear yellow liquid w/ some white residue |

The supernatant was collected from each sample and assayed for the Simvastatin content by HPLC (% label claim, (% LC)). Table 5 provides results of the assays.

TABLE 5
Assay Results.

| Sample Identity | Assay (% LC) |
|---|---|
| Propylene Glycol | 101.1 |
| Glycerine | 40.7 |
| Labrasol | 105.3 |
| Captex 355 | 95.3 |
| E. Pure Water | 0.3 |
| Labrafil M 2125 CS | 104.1 |
| Captex 500 P | 114.1 |
| PEG 400 | 108.7 |
| Polysorbate 80 | 72.8 |
| 5% SLS in Water | 86.1 |

The visual observation and the assay data indicates that the solubility of Simvastatin was achieved at 2.5 mg/g in six of the ten vehicles employed. The data indicate that E. pure water and glycerine are not viable vehicles due to having the least solubility. Polysorbate 80 and 5% SLS in water demonstrated solubility to an extent and may be considered in smaller quantities for initial wetting purposes, if required.

### Prototype Development

Prototype formulations were prepared with mixtures of vehicles from the above solubility screening, and preservatives and sweetener also were incorporated as part of the matrix. Table 6, below, provides descriptions of initial prototype batches that were prepared.

TABLE 6
Prototype Development of Simvastatin at 2.0 mg/g.

| Batch # | Components | % w/w |
|---|---|---|
| N2482-9A | Simvastatin | 0.2 |
| | Propylene Glycol | 59.8 |
| | Methylparaben | 1.0 |
| | Propylparaben | 0.5 |
| | Sorbitol | 10 |
| | Water | 28.5 |
| N2482-17A | Simvastatin | 0.2 |
| | Captex ® 500 P | 59.8 |
| | Methylparaben | 0.2 |
| | Propylparaben | 0.02 |
| | Sorbitol | 11.25 |
| | Water | 28.53 |
| N2482-17C* | Simvastatin | 0.2 |
| | PEG 400 | 10 |
| | Captex ® 500 P | 50 |
| | Methylparaben | 0.2 |
| | Propylparaben | 0.02 |
| | Sorbitol | 11.05 |
| | Water | 28.53 |

*Batch was not completed due to the vehicles being immiscible once mixed together.

The following batches also were prepared at the same concentrations as in Batch N2482-9A, except that propylene glycol was substituted with an alternative vehicle for each batch as follows: Batch N2482-9B substituted Labrasol; Batch N2482-9C substituted Labrafil M 2125 CS; and Batch N2482-9D substituted Captex® 355. Batch N2482-17B was prepared using the same vehicles and concentrations as Batch N2482-17A, except that PEG 400 was substituted for Captex® 500 P in Batch N2482-17B. The following batches also were prepared at the same concentrations as in Batch N2482-17C, except that PEG 400/Captex® 355 was substituted with the following vehicles: in Batch 2482-17D, PEG 400/Labrasol was substituted; in Batch 2482-17E, PEG 400/Labrofil M 2125 CS was substituted.

Table 7 provides pH and observation data for the prototype batches described above.

TABLE 7
Observations of Batches N2482-9 Series.

| | pH of API | | Final |
|---|---|---|---|
| Batch # | Solution | Final Solution Observation | pH |
| N2482-9A | 5.19 | Clear colorless liquid; ~4-5 particles; | 4.04 |
| | | ~5 fibrous pieces of material | |
| N2482-9B | 4.93 | 2 layers; top layer was a clear pale | 3.79 |
| | | yellow liquid; bottom layer was a clear | |
| | | and colorless liquid; upon mixing, an | |
| | | opaque off-white liquid was observed. | |
| N2482-9C | 6-7 | Pale off-white milky liquid upon | 4.01 |
| | (reading | mixing; upon standing, top layer was | |
| | fluctuated) | an off-white opaque milky liquid | |
| | | and bottom layer was an opaque | |
| | | white liquid | |
| N2482-9D | 12.13 | 2 layers; top layer was a clear colorless | 2.86 |
| | | oily (oil droplets) liquid; bottom layer | |
| | | was a clear colorless liquid | |
| N2482-17A | 9.49 | 2 phase liquid (oil and water), oil | 3.82 |
| | | droplets were present | |
| N2482-17B | 5.21 | Clear colorless liquid with air bubbles | 3.39 |
| N2482-17C | * | * | * |
| N2482-17D | 6.97 | Hazy liquid | 3.20 |
| N2482-17E | — | Opaque pale yellow liquid w/ oil | 3.71 |
| | | droplets; milky yellow liquid upon | |
| | | mixing | |

* Not completed. Vehicles were immiscible.

Batches N2482-9A and N2482-17B were submitted for assay analysis as these were the only two batches observed to be visually clear colorless solutions. Results of the chemical testing performed on the clear intact one phase visual systems are provided in Table 8.

TABLE 8
Assay Results of Prototypes.

| Batch # | Assay (% Label Claim) |
|---|---|
| N2482-9A | 107.7 |
| N2482-17B | 115.2 |

### Manufacturing Process:

For each batch, Simvastatin was solubilized in the vehicle with mixing and the pH of the API solution was recorded. Methyl and propylparaben were transferred to the API solution with mixing. Sorbitol was dissolved in water and added to the API solution while mixing continued.

Initially, when batch N2482-9A was prepared methylparaben (1% w/w) was dissolved in water. However, the methylparaben never dissolved. Heat was applied and the methylparaben dissolved but precipitated upon cooling. Due to the precipitation and not wanting to apply heat, the methylparaben concentration was reduced to 0.5% in all of the N2482-9 batch series while the propylparaben's concentration was reduced to 0.2%.

Proceeding with the N2482-17 batch series, the methylparaben and propylparaben concentrations were decreased to 0.2% and 0.02%, respectively, and were also soluble in the vehicles for each batch.

### Example 2

### Stability of Prototype Liquid Formulations of Simvastatin

Prototype batches N2482-9A and N2482-17B, see Example 1, were re-assayed to evaluate the stability of the prototypes held at room temperature (R.T.). Assay results are shown below in Table 9.

TABLE 9
Assay Results for Prototypes.

| Tests | | Batch N2482-9A | Batch N2482-17B |
|---|---|---|---|
| Assay | Initial | 107.7 | 115.2 |
| (% Label Claim) | ~2 weeks | 103.8 | 97.7 |
| pH | Initial | 4.04 | 3.39 |
| | ~1 week | 3.82 | 3.77 |
| | ~2 weeks | 4.58 | 4.86 |

Based on the fluctuation in the pH of batches N2482-9A and N2482-17B, two additional prototypes, batches N2482-28A and N2482-28B, were prepared with and without sodium benzoate (an alternate preservative), and by combining propylene glycol and PEG 400 in a 1:1 ratio as the vehicle to solubilize the simvastatin. Sodium benzoate was considered in the formulation to increase the pH in lieu of obtaining a pH between 4-7. Table 10 provides the component vehicles and simvastatin concentration in % w/w.

TABLE 10
Prototype Development of a 2 mg/g Simvastatin Solution.

| | Batch N2482-28A | Batch N2482-28B |
|---|---|---|
| Components | % w/w | % w/w |
| Simvastatin | 0.2 | 0.2 |
| Propylene Glycol | 30 | 30 |
| PEG 400 | 30 | 30 |
| Methylparaben | 0.2 | 0.2 |
| Propylparaben | 0.02 | 0.02 |
| Sodium benzoate | — | 0.5 |
| Sorbitol | 10 | 10 |
| Water | 29.58 | 29.08 |

The results of assay testing of these two batches at their initial time point and at approximately 2 week later are shown in Table 11 below. Based on these results, the combination of the two parabens remained the preservative system of choice as a result of the sodium benzoate decreasing % LC and the pH being inconsistent.

TABLE 11
Assay Results.

| | |
|---|---|
| Assay (% LC) | pH |

| Batch # | Initial | ~2 weeks | Initial | ~2 weeks |
|---|---|---|---|---|
| N2482-28A | 100.2 | 87.7 | 3.47 | 4.80 |
| N2482-28B | 82.0 | 59.8 | 7.30 | 6.74 |

Next, formulations the incorporation of the anti-oxidant sodium metabisulfite and each of three flavors (bubble gum, cherry, and grape) were examined. Table 12 provides these prototype formulations. Upon final admixing, all prototype batches were clear colorless liquids containing a few pieces of fibrous material.

TABLE 12
Prototypes with Sodium Metabisulfite and Flavors.

| | Batch | Batch | Batch |
|---|---|---|---|
| | N2482-47A | N2482-47B | N2482-47C |
| Components | % w/w | % w/w | % w/w |
| Simvastatin | 0.2 | 0.2 | 0.2 |
| Propylene Glycol | 59.8 | — | 30 |
| PEG 400 | — | 59.8 | 30 |
| Methylparaben | 0.5 | 0.2 | 0.2 |
| Propylparaben | 0.2 | 0.02 | 0.02 |
| Sodium metabisulfite | 0.1 | 0.1 | 0.1 |
| Sorbitol | 10 | 10 | 10 |
| Water | 29.2 | 29.68 | 29.48 |
| | Batch | Batch | Batch |
| | N2482-47A-1 | N2482-47B-1 | N2482-47C-1 |

| | |
|---|---|
| Bubble Gum | 0.1% |
| Cherry Grape | 0.1% |
| Grape | 0.1% |

Each of these flavored batches were submitted for assay and results are shown in Tables 13 and 14 below. The results were not as predicted. The addition of flavor reduced the pH in all of the batches. Investigational batches were manufactured to determine what could have caused the low assay results. Sodium metabisulfite (anti-oxidant) and the flavors were not used in the previous prototypes. Sodium metabisulfite was chosen to use due to it being soluble in water. The flavors were expected to have been compatible since the dissolved solvent in each flavor is propylene glycol. Further, the study plan included assay testing of aliquots of the batches (results shown in Table 15 below).

TABLE 13
pH of Prototype Solutions Containing 2 mg/g Simvastatin.

| Tests | Batch 2482-47A | | | Batch 2482-47B | | | Batch 2482-47C | | |
|---|---|---|---|---|---|---|---|---|---|
| pH after | 6.24 | | | 7.60 | | | 6.52 | | |
| solubilizing | | | | | | | | | |
| simvastatin | | | | | | | | | |
| Final pH | 6.28 | | | 6.07 | | | 6.36 | | |
| | Batch | Batch | Batch | Batch | Batch | Batch | Batch | Batch | Batch |
| | N2482- | N2482- | N2482- | N2482- | N2482- | N2482- | N2482- | N2482- | N2482- |
| | 47A1 | 47A2 | 47A3 | 47B1 | 47B2 | 47B3 | 47C1 | 47C2 | 47C3 |
| Final pH | 5.89 | 5.49 | 5.20 | 5.56 | 5.54 | 5.61 | 5.93 | 5.81 | 5.77 |
| (after | | | | | | | | | |
| addition of | | | | | | | | | |
| flavor) | | | | | | | | | |

TABLE 14
Assay of Batches Listed Table 13.

| Batch # | Assay (% LC) |
|---|---|
| N2482-47A1 | 35.5 |
| N2482-47A2 | 45.4 |
| N2482-47A3 | 40.6 |
| N2482-47B1 | 27.6 |
| N2482-47B2 | 31.1 |
| N2482-47B3 | 48.4 |
| N2482-47C1 | 49.3 |
| N2482-47C2 | 55.3 |
| N2482-47C3 | 49.2 |

TABLE 15
Assay Results.

| Batch # | Description | Assay (% LC) |
|---|---|---|
| N2498-12A1 | API solubilized in 1:1 vehicle solution of | 109.7 |
| | propylene glycol and PEG 400 | |
| N2498-12A2 | API/vehicle solution with parabens and | 35.6 |
| | sorbitol/sodium metabisulfite solution | |
| N2498-12A3 | API/vehicle solution with parabens and | 22.4 |
| | sorbitol/sodium metabisulfite solution and | |
| | bubble gum flavor | |
| N2498-12B | API/vehicle solution with parabens and | 110.2 |
| | sorbitol solution, and bubble gum flavor | |
| N2498-15P | Placebo solution | ND* |

*ND—none detected.

Based on these assay results, the batches containing the sodium metabisulfite assayed low, suggesting that this particular anti-oxidant caused degradation. Assay of batch N2498-12A1 verified that the API was completely soluble in the vehicle and was not degrading. The assay results obtained in Table 15 for batches N2498-A2 and A3, respectively, corresponded with the assay results obtained in Table 14. The flavor did not cause degradation as observed in the assay value of batch N2498-12B in Table 15. Thus, alternative anti-oxidants were sourced for the development a simvastatin solution as detailed in Example 3 below.

### Example 3

### Prototype Development with Preservative and Anti-Oxidant Systems

The results of Examples 1 and 2 demonstrate that development of liquid solutions containing therapeutic amounts of simvastatin and suitable for consumption is not easily predicted. Further, the addition of preservatives and flavors can affect suitability of the liquid solution. Below is a summary of the compositions (Tables 16a and 16b) of simvastatin solutions that were prepared using the methods described in Examples 1 and 2.

TABLE 16a
Prototype Development with Preservative and Antioxidant Combinations.

| | % w/w per Batch # | | | | | | |
|---|---|---|---|---|---|---|---|
| | N2482- | N2482- | N2498- | N2498- | N2498- | N2498- | N2498- |
| Factors | 67E | 78A | 29A | 29C | 29D | 29D2 | 29E |
| Simvastatin | 0.2 | 0.2 | 0.2 | 0.2 | 0.2 | 0.2 | 0.2 |
| Propylene | 30 | 30 | — | 59.8 | — | — | — |
| Glycol | | | | | | | |
| PEG 400 | 30 | 30 | — | — | 59.8 | 59.8 | 59.8 |
| Cremophor EL¹ | — | — | — | — | — | — | — |
| Labrasol | — | — | 25 | — | — | — | — |
| Polysorbate 80 | — | — | 0.25 | — | — | — | — |
| Methylparaben | — | 0.2 | — | — | 0.2 | 0.2 | — |
| Propylparaben | — | 0.02 | — | — | 0.02 | 0.02 | — |
| Sodium | 1.0 | — | 1.0 | 1.0 | — | — | 1.0 |
| benzoate | | | | | | | |
| BHA² | 0.01 | 0.01 | 0.01 | 0.01 | 0.01 | 0.01 | 0.01 |
| BHT³ | — | 0.01 | — | — | — | — | — |
| Glycerine | — | 10 | — | — | — | — | — |
| Citric acid | 0.5 | — | 0.58 | 0.89 | 0.01 | — | 1.8 |
| Sorbitol | 10 | 10 | 10.0 | 10 | 10 | 10 | 10 |
| Grape flavor | 0.1 | 0.1 | 0.1 | 0.1 | 0.1 | 0.1 | 0.1 |
| Water | 29.69 | 19.46 | 63.34 | 28.79 | 29.57 | 29.57 | 28.79 |

¹PEG 35 Castor oil
²Butylated hydroxyanisole
³Butylated hydroxytoluene

TABLE 16b
Prototype Development with Preservative
and Antioxidant Combinations.

| | % w/w per Batch # | | | | | |
|---|---|---|---|---|---|---|
| | N2498- | N2498- | N2498- | N2498- | N2498- | N2498- |
| Factors | 35A | 41A | 41A2 | 41B | 41C | 51A |
| Simvastatin | 0.2 | 0.2 | 0.2 | 0.2 | 0.2 | 0.2 |
| Propylene | 30 | 59.8 | 59.8 | — | — | 59.8 |
| Glycol | | | | | | |
| PEG 400 | 30 | — | — | 59.8 | — | — |
| Cremophor | — | — | — | — | 35 | — |
| EL¹ | | | | | | |
| Labrasol | — | — | — | — | — | — |
| Polysorbate 80 | — | — | — | — | — | — |
| Methylparaben | 0.2 | — | — | 0.2 | 0.2 | 0.2 |
| Propylparaben | 0.02 | — | — | 0.02 | 0.02 | 0.02 |
| Sodium | — | 1.0 | 1.0 | — | — | — |
| benzoate | | | | | | |
| BHA² | 0.01 | — | — | — | — | — |
| BHT³ | 0.01 | 0.01 | 0.01 | 0.01 | 0.01 | 0.01 |
| Glycerine | 39.46 | 10 | 10 | 10 | 10 | 39.67 |
| Citric acid | — | 0.1 | 0.85 | — | — | — |
| Sorbitol | — | 10 | 10 | 10 | 10 | — |
| Grape flavor | 0.1 | 0.1 | 0.1 | 0.1 | 0.1 | 0.1 |
| Water | — | 18.79 | 18.79 | 19.67 | 44.47 | — |

¹PEG 35 Castor oil
²Butylated hydroxyanisole
³Butylated hydroxytoluene

Analyses of these simvastatin solutions were conducted. In particular, preservative and anti-oxidant systems, and stability were evaluated. See Tables 17a, 17b, 18a, and 18b below. Based on these results, preferred simvastatin oral solutions for treating children or adolescents are Batch numbers N2498-29D2, N2498-35A, N2498-41B, N2498-51B, and N2498-51C. Batches N2498-29D2, N2498-35A, and N2498-41B are the most preferred simvastatin oral solutions.

TABLE 17a
pH of Prototype Development Batches.

| | N2482- | N2482- | N2498- | N2498- | N2498- | N2498- | N2498- |
|---|---|---|---|---|---|---|---|
| Tests | 67E | 78A | 29A | 29C | 29D | 29D2 | 29E |
| pH, | 4.65 | 5.09 | 4.86 | 4.92 | 4.08 | 4.30 | 4.41 |
| R.T.* | (Initial) | (Initial) | (Initial) | (Initial*) | (Initial) | (Initial) | (Initial) |
| | 4.44 | 4.31 | 4.83 | 4.80 | 4.44** | 5.04 | |
| | (Day 3) | (Day 24) | (24 Hrs) | (Day 2) | (Day 4) | (Day 6) | |
| | 4.59 | 4.36 | | 4.65 | 4.51** | 5.18 | |
| | (Day 4) | (Day 2) | | (Day 5) | (Day 6) | (Day 7) | |
| | 4.47 | 4.13 | | 4.65 | 4.60** | 4.43 | |
| | (Day 5) | (Day 7) | | (Day 7) | (Day 7) | (Day 11) | |
| | 4.51 | 5.15 | | 4.86 | | 4.68** | |
| | (Day 7) | (Day 9) | | (Day 8) | | (Day 18) | |
| | | | | 4.39 | | | |
| | | | | (Day 12) | | | |
| pH, | — | — | 4.86^{a} | 4.59 | 4.85^{1,} ** | 4.86³ | 4.56³ |
| 40° C. | | | (Day 5) | (24 Hrs) | (Day 6) | (Day 4) | (day 4) |
| | | | | 4.82 | 4.76² | 5.44⁴ | |
| | | | | (Day 2) | (Day 7) | (Day 6) | |
| | | | | 4.43 | | 5.43⁵ | |
| | | | | (Day 10) | | (Day 7) | |
| | | | | | | 4.70 | |
| | | | | | | (Day 10) | |
| | | | | | | 4.89 | |
| | | | | | | (day 17) | |
| pH, | — | — | — | 4.65 | 4.60⁶ | 5.35^{8,} ** | — |
| 2-8° C. | | | | (24 Hrs) | (Day 6) | (Day 6) | |
| | | | | 4.74¹⁰ | 4.64⁷ | 5.28⁹ | |
| | | | | (Day 2) | (Day 7) | (Day 7) | |
| | | | | 4.45 | | 4.92 | |
| | | | | (Day 10)¹¹ | | (Day 17) | |

*pH was lowered to 4.54.
**Crystals present.
^{a}3 of 5 days at 40° C.
¹24 hours at 40° C.
²2 days at 40° C.
³3 days at 40° C.
⁴5 days at 40° C.
⁵6 days at 40° C.
⁶24 hrs at 2-8° C.; cloudy liquid.
⁷2 days at 2-8° C.
⁸24 hrs at 2-8° C.
⁹6 days at 2-8° C.
¹⁰Some particles present.
¹¹Colorless liquid with particles.

TABLE 17b
pH of Prototype Development Batches.

| | N2498- | N2498- | N2498- | N2498- | N2498- | N2498- |
|---|---|---|---|---|---|---|
| Tests | 35A | 41A | 41A2 | 41B | 41C | 51A |
| pH, | 5.95 | 6.35 | 4.84 | 4.52 | 7.14 | 4.99 |
| R.T.* | (Initial) | (Initial) | (Initial) | (Initial) | (Initial) | (Initial) |
| | 6.09 | 6.03 | 4.47 | 4.16 | 6.26 | 5.20 |
| | (Day 2) | (Day 4) | (Day 4) | (Day 4) | (Day 4) | (Day 5) |
| | 5.77 | | | 4.64 | 6.09 | |
| | (Day 3) | | | (Day 11) | (Day 11) | |
| | 4.91 | | | | | |
| | (Day 7) | | | | | |
| | 4.92 | | | | | |
| | (Day 14) | | | | | |
| pH, | 6.12 | — | 4.49 | 4.24 | 5.83 | 5.20 |
| 40° C. | (Day 2) | | (Day 4) | (Day 4) | (Day 4) | (Day 5) |
| | 5.92 | | | 4.49 | 5.77 | |
| | (Day 3) | | | (Day 11) | (Day 11) | |
| | 5.15 | | | | | |
| | (Day 7) | | | | | |
| | 5.19 | | | | | |
| | (Day 14) | | | | | |
| pH, | 6.02 | — | 4.58 | 4.28 | 6.35 | 5.28 |
| 2-8° C. | (Day 2) | | (Day 4) | (Day 4) | (Day 4) | (Day 5) |
| | 5.83 | | | 4.59 | 6.39 | |
| | (Day 3) | | | (Day 11) | (Day 11) | |
| | 5.15 | | | | | |
| | (Day 7) | | | | | |
| | 5.28 | | | | | |
| | (Day 14) | | | | | |

*pH was lowered to 4.54.

TABLE 18a
Assay of Prototype Development Batches.

| | N2482- | N2482- | | N2498- | N2498- | | N2498- |
|---|---|---|---|---|---|---|---|
| Tests | 67E | 78A | N2498-29A | 29C | 29D | N2498-29D2 | 29E |
| Assay | 101 | 113.4 | 99.6 | 98.3 | 104.5 | 109.3 | 107 |
| (% | (Initial at | (Initial at | (Initial at | (Initial at | (Initial at | (Initial at | (Initial) |
| LC) | RT) | RT) | RT) | RT) | RT) | RT) | 80.6 |
| | 82.2 | 111.7 | 74.7 | 97.3 | 105.8 | 110.7 | (Day 4) |
| | (Day 6 | (Day 2 | (Day 5; 3 of | (Day 5 at | (Day 4 | (Day 4; 3 of | |
| | at RT) | at RT) | 5 days at | RT) | at RT) | 4 days at | |
| | | 105.9 | 40° C.) | 85.7 | | 40° C.) | |
| | | (Day 6 | | (Day 9 at | | 108.6 | |
| | | at RT) | | RT) | | (Day 8 at | |
| | | | | 69.0 | | RT) | |
| | | | | (Day 3 at | | 110.7 | |
| | | | | 40° C.) | | (Day 7 at | |
| | | | | 90.0 | | 40° C.) | |
| | | | | (Day 3 at | | 132.5 | |
| | | | | 2-8° C.) | | (Day 7 at 2-8° C.) | |
| | | | | | | 103 | |
| | | | | | | (Day 14 at | |
| | | | | | | 40° C.) | |

TABLE 18b
Assay of Prototype Development Batches.

| | N2498- | N2498- | N2498- | N2498- | N2498- | |
|---|---|---|---|---|---|---|
| Tests | 35A | 41A | 41A2 | 41B | 41C | N2498-51A |
| Assay | 119.5 | 107.3 | 104.2 | 114.1 | 105.0 | 107.4 |
| (% LC) | (Initial at | (Initial | (Initial | (Initial | (Initial | (Initial at |
| | RT) | at RT) | at RT) | at RT) | at RT) | RT, assayed |
| | 111.7 | | | 97.7 | 87.4 | 2 days after |
| | (Day 4 at | | | (Day 11 | (Day 11 | manufacture) |
| | 40° C.) | | | at | at | |
| | 104.9 | | | 40° C.) | 40° C.) | |
| | (Day 14 | | | | | |
| | at 40° C.) | | | | | |
| | 105.1 | | | | | |
| | (Day 14 | | | | | |
| | at 2-8° C.) | | | | | |
| | 110.8 | | | | | |
| | (Day 14 | | | | | |
| | at RT) | | | | | |

### Example 4

### Other Statin Liquid Solutions

It is expected that liquid formulations containing statins other than simvastatin may also be prepared using the procedures described herein. For example, it is expected that liquid formulations of provastatin, atorvastatin, or pitavastatin can be prepared by substituting one of these statins (or a combination of two or more of them) for simvastatin in any of the formulas N2482-78A or N2498-29D2 (see Table 16a) or N2498-35A, N2498-41B, or N2498-51A (see Table 16b). Preferably, in each of these formulations, the chosen statin would be at a concentration of about 2 mg/ml.

It is also expected that a combination of simvastatin with provastatin, atorvastatin, or pitavastatin can also be prepared using these formulations. Preferably, such combinations would include a total statin concentration of about 2 mg/ml where the amount of simvastatin would range from 0.01-1.99 mg/ml, but more preferably would be about 0.05-1.5 mg/ml.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the following claims.

### REFERENCES

The following references, to the extent that they provide exemplary procedural or other details supplementary to those set forth herein, are specifically incorporated herein by reference.
U.S. Pat. No. 4,444,784.
U.S. Pat. No. 4,450,171.
Avis H J, Vissers M N, Stein E A, Wijburg F A, Trip M D, Kastelein J J et al. A systematic review and meta-analysis of statin therapy in children with familial hypercholesterolemia. Arterioscler Thromb Vasc Biol 2007; 27(8):1803-1810.
Dirisamer et al., The effect of low-dose simvastatin in children with familial hypercholesterolaemia: a 1-year observation. Eur J Pediatr 2003; 162(6): 421-425.
Ducobu et al., Simvastatin use in children, Lancet 1992; 339(8807):1488.
Knipscheer H C, Boelen C C, Kastelein J J, van Diermen D E, Groenemeijer B E, van den E A et al. Short-term efficacy and safety of pravastatin in 72 children with familial hypercholesterolemia. Pediatr Res 1996; 39(5):867-871.
Lambert M, Lupien P J, Gagne C, Levy E, Blaichman S, Langlois S et al. Treatment of familial hypercholesterolemia in children and adolescents: effect of lovastatin. Canadian Lovastatin in Children Study Group. Pediatrics 1996; 97(5):619-628.
Travia D, Tosi F, Negri C, Faccini G, Moghetti P, Muggeo M. Sustained therapy with 3-hydroxy-3-methylglutaryl-coenzyme-A reductase inhibitors does not impair steroidogenesis by adrenals and gonads. J Clin Endocrinol Metab 1995; 80(3):836-840.

1. A liquid solution comprising 0.05-10% w/w of a statin and at least one solubilizer selected from the group consisting of propylene glycol, minerals, propylene glycolmonostearate, propylene glycol alginate, natural glycerine, niacin, synthetic glycerine, vitamins, sorbitol, alcohols, myristyl alcohol, carboxymethylcellulose, labrasol, copovidone, Captex 355, croscarmellose sodium, polyethylene glycol (PEG) 400, PEG 1000, PEG 1450, PEG 1540, crospovidone, ethyl cellulose, aqueous polysorbate 20, aqueous polysorbate 40, aqueous polysorbate 60, aqueous polysorbate 80, cellulose, oxidized cellulose, polyoxyl 10 oleoyl ether, cellulose sodium phosphate, polyoxyl 20 cetostearyl, hyopromellose, poloyxyl 35 castor oil, polyoxyl 40 hydrogentated castor oil, polyoxyl 40 stearate, poloxyl lauryl ether, poloxyl oleate, poloxyl stearyl ether, and any combination thereof.

2. The liquid solution of claim 1 wherein the statin is selected from the group consisting of simvastatin, lovastatin, pravastatin, atorvastatin, pitavastatin, mevastatin, cerivastatin, fluvastatin, and rosuvastatin.

3. The liquid solution of claim 1 further comprising at least one of the group consisting of an antioxidant, a flavoring, a preservative, and any combination thereof.

4. The liquid solution of claim 3 further comprising at least one of the group consisting of an amino acid, a vitamin, mineral, phospholipid, cyclodextrin, triglyceride, diglyceride, monoglyceride, surfactant, bile salt, fatty acid, sweetener, buffer, and any combination thereof.

5. The liquid solution of claim 3 wherein the antioxidant is selected from the group consisting of butylated hydroxytoluene (BHT), butylated hydroxyanisole (BHA), and combinations thereof.

6. The liquid solution of claim 3 wherein the flavoring is selected from the group consisting of grape syrup, grape cherry syrup, bubble gum, almond oil, anise oil, cherry syrup, clove oil, lemon oil, licorice fluid extract, orange oil, orange syrup, peppermint oil, vanilla tincture, and combinations thereof.

7. The liquid solution of claim 3 wherein the preservative is selected from the group consisting of propylparaben, methylparaben, methylparaben sodium, and combinations thereof.

8. The liquid solution of claim 4 wherein the phospholipid is selected from the group consisting of phosphotidyl choline, phophotidyl ethanolamine, sphingomylein, lauroyl polyoxylglyceride, linoleoyl polyoxylglyceride, oleoyl polyoxylglyceride, lecithin, soy isoflavones, and combinations thereof.

9. The liquid solution of claim 4 wherein the cyclodextrin is selected from the group consisting of α cyclodextrin, β cyclodextrin, δ cyclodextrin, γ cyclodextrin, and combinations thereof.

10. The liquid solution of claim 4 wherein the triglyceride is selected from the group consisting of olive oil, safflower oil, soybean oil, sunflower oil, and combinations thereof.

11. The liquid solution of claim 4 wherein the bile salt is cholesterol.

12. The liquid solution of claim 4 wherein the fatty acid is selected from the group consisting of oleic acid, stearic acid, α-lipoic acid, ethyl oleate, myristic acid, palmitic acid, sorbitan monolaurate, sorbitan monooleate, sorbitan monopalmitate, sorbitan monostearate, sorbitan trioleate, and combinations thereof.

13. The liquid solution of claim 4 wherein the amino acid is selected from the group consisting of alanine, arginine, aspartic acid, choline, folic acid, glutamine, glycine, histidine, isoleucine, leucine, lysine, methionine, phenylalanine, serine, threonine, tryptophan, tyrosine, valine, and combinations thereof.

14. The liquid solution of claim 4 wherein the sweetener is selected from the group consisting of aspartame, calcium saccharate, dextrose, fructose, maltodextrin, maltose, mannitol, polydextrose, potassium sorbate, saccharin, saccharin calcium, saccharin sodium, sorbitol, sucralose, sucrose, sugar, xanthane gum, xylitol, xylose, and combinations thereof.

15. The liquid solution of claim 4 wherein the buffer is selected from the group consisting of ascorbic acid, ascorbyl palmitate, calcium sulfate, citric acid, dibasic sodium phosphate, monobasic sodium phosphate, potassium carbonate, potassium citrate, sodium acetate, sodium ascorbate, sodium bicarbonate, sodium carbonate, sodium citrate, sodium lauryl sulfate, sodium metabisulfate, and combinations thereof.

16. The liquid solution of claim 4 wherein the surfactant is selected from the group consisting of aqueous sodium laurel sulfate, a tocopherol excipient, tocopherol polyethyleneglycol, beta-carotene, lycopene, and combinations thereof.

17. A liquid formulation comprising weight to weight (w/w) 0.2% simvastatin, 38.47% polyethylene glycol (PEG) 400, 0.2% methylparaben, 0.02% propylparaben, 0.01% butylated hydroxytoluene, 60% glycerine, 0.1% flavoring, and 1.0% saccharin sodium.

18. The liquid formulation according to claim 17, further comprising 0.15% a second flavoring, and 0.1% a third flavoring.

19. (canceled)

20. (canceled)

21. (canceled)

22. Use of a liquid solution in treating high cholesterol comprising oral administration to an individual of the liquid solution of claim 1.

23. The use of claim 22 wherein the individual is a child, adolescent, or animal.

24. (canceled)

25. (canceled)
